(19) United States
(12) Patent Application Publication
(10) Pub. No.: US 2007/0212390 A1
(43) Pub. Date: Sep. 13, 2007

(54) Protease-resistant forms of VEGF-D, method of making and method of use

(75) Inventors: Karri Paavonen, Melbourne (AU); Steven Stacker, Melbourne (AU); Marc G. Achen, Melbourne (AU)

Correspondence Address: David A. Gass;Marshall Gerstein & Borun, LLP, Sears Tower - Suite 6300, 233 South Wacker Drive, Chicago, IL, 60606, US

(73) Assignee: Ludwig Institute for Cancer Research, New York, NY (US)

(21) Appl. No.: 11/517,290

(22) Filed: Sep. 8, 2006

Related U.S. Application Data
(60) Provisional application No. 60/719,184, filed on Sep. 22, 2005.

(51) Int. Cl. A61K 38/18; C07K 14/475; C07H 21/04; C12P 21/06

(52) U.S. Cl. 424/423; 514/12; 530/399; 435/69.1; 435/320.1; 435/325; 435/366; 435/455; 536/23.5

(57) ABSTRACT

The present invention provides modified VEGF-D polypeptide variants that are resistant to serine protease processing and methods of making and using the same, as well pharmaceutical compositions comprising the peptide or a polynucleotide encoding the same. The VEGF-D variants comprise a VEGF homology domain, and at least one of (1) a C-terminal propeptide that is not cleavable by a serine protease, and (2) an N-terminal propeptide that is not cleavable by a serine protease. The VEGF-D variants can be made using site-directed mutagenesis. The VEGF-D variants are useful for the treatment of diseases such as cardiovascular disease and primary and secondary lymphedema, and for the prevention of stenosis and restenosis of blood vessels.

### CROSS-REFERENCE TO RELATED APPLICATION

This application claims priority of U.S. Application Ser. No. 60/719,184 filed Sep. 22, 2005, the disclosure of which is incorporated by reference herein in its entirety.

### BACKGROUND OF THE INVENTION

Vascular Endothelial Growth Factor (VEGF)-D is a member of the VEGF family of growth factors, which include VEGF, Placenta Growth Factor (PlGF), VEGF-B, VEGF-C, and VEGF-D (Tammela T. et al., Cardiovasc. Res. 65 (2005) 550-563). VEGF-D binds to and activates the tyrosine kinase receptors VEGF Receptor-2 (VEGFR-2) and VEGFR-3 on the surface of endothelial cells (Achen M. G. et al., Proc. Natl. Acad. Sci. USA 95 (1998) 548-553). VEGFR-2 and VEGFR-3 are intimately involved in the growth of novel blood vessels from pre-existing vessels (angiogenesis) and of novel lymphatic vessels (lymphangiogenesis), respectively (Veikkola T. et al., Cancer Res. 60 (2000) 203-212). These processes are involved in cancer growth and metastasis, e.g. hematogenous metastasis via blood vessels and lymphogenous metastasis via lymphatic vessels. Recent advances in drug development have seen the launch into the clinic of the first anti-angiogenic agent, Avastin (bevacizumab), an anti-VEGF antibody for treatment of metastatic cancer (Yang J. C. et al., New Engl. J. Med. 349 (2003) 427-434; Kabbinavar F. et al., J. Clin. Oncol. 21 (2003) 60-65). Lymphangiogenesis research also offers the opportunity for developing novel agents to restrict the spread of cancer and has advanced during the past decade with the discovery of VEGF-C, VEGF-D, the lymphatic endothelial cell-specific receptor VEGFR-3 and other markers for lymphatic endothelium. In addition to inhibiting angiogenesis and lymphangiogenesis in the setting of cancer and other diseases, clinical benefit could be derived by using growth factors such as VEGF-C and VEGF-D to promote angiogenesis and lymphangiogenesis in the setting of ischaemic heart disease, peripheral arterial disease/claudication and lymphedema (Saharinen P. et al., Trends Immunol. 25 (2004) 387-395).

In humans, VEGF-D is initially secreted as a homodimer with subunit molecular mass of approximately 50 kDa. This protein is subsequently proteolytically processed with cleavage of the N-terminal propeptide of 10 kDa and of the C-terminal propeptide of 29 kDa from each subunit of the dimer to yield a fully activated mature form consisting of dimers of the central 21 kDa VEGF homology domain (VHD) (Stacker S. A. et al., J. Biol. Chem. 274 (1999) 32127-32136). A recombinant version of this mature form, tagged at the N-terminus of each subunit with the FLAG octapeptide, was previously designated VEGF-DΔNΔC. The proteolytic processing of VEGF-D is extremely important for the interaction with receptors as VEGF-DΔNΔC binds VEGFR-2 with a 290-fold higher affinity than does the full-length form. Further, VEGF-DΔNΔC binds VEGFR-3 with a 40-fold higher affinity than does full-length VEGF-D (Stacker S. A. et al., J. Biol. Chem. 274 (1999) 32127-32136). The dramatic alteration in receptor affinity upon proteolytic processing, particularly with respect to the angiogenic receptor VEGFR-2, indicates that this processing is a critical regulator of the bioactivity of VEGF-D. This is supported by the observation that full-length VEGF-D promoted lymphangiogenesis, but not angiogenesis, in vivo in a rabbit muscle model whereas mature, fully-processed VEGF-D promoted both lymphangiogenesis and angiogenesis in the same model (Rissanen T. T. et al., Circ. Res. (92) 2003 1098-1106).

VEGF-D is proteolytically processed by several enzymes, namely plasmin and members of the proprotein convertase family of proteases including furin, PC5 and PC7 (McColl B. K. et al., J. Exp. Med. 198 (2003) 863-868 and U.S. patent Ser. No. 11/133,415). Cleavage by furin is a means by which to regulate the biological activity of many proteins. Furin is a serine protease that is very broadly expressed in human physiology and activates a range of proproteins including insulin-like growth factor and some matrix metalloproteinases. Furin cleaves its target peptides after the consensus site Arginine (R)-X-Lysine/Arginine (K/R)-Arginine (R) or the alternative site Arginine (R)-Xₙ-Arginine (R) (where n=0, 2, 4 or 6 amino acid residues). The processing of VEGF-D is similar in nature to the processing of its most closely related protein VEGF-C (Siegfried G. et al., J. Clin. Invest. 111 (2003) 1723-1732). Stepwise proteolytic processing of VEGF-C generates several forms with increasing activity towards VEGFR-3, but only the fully processed form activates VEGFR-2 (Joukov V. EMBO J. 16 (1997) 3898-3911).

VEGF-D has been shown to increase tumor growth and lymphatic metastasis in a murine model utilizing a human xenograft tumor (Stacker S. A. et al., Nat. Med. 7 (2001) 186-191). Further, mature VEGF-D, VEGF-DΔNΔC, induces both angiogenesis and lymphangiogenesis when delivered as a gene therapeutic agent with adenovirus (Rissanen T. T. et al., Circ. Res. (92) 2003 1098-1106). Intramyocardial delivery of VEGF-DΔNΔC induced strong angiogenesis in the porcine myocardium (Rutanen J. et al., Circulation 109 (2004) 1029-1035). Similar delivery of VEGF-DΔNΔC into the denuded aorta of the rabbit resulted in decreased intimal thickening through a nitric oxide synthase dependant fashion (Rutanen J. et al., Gene Ther. 12 (2005) 980-987). These studies have shown the potency of VEGF-D as a gene therapeutic agent for treating several clinical conditions such as peripheral arterial disease/claudication and coronary heart disease and for inhibition of re-stenosis of angioplasty-treated coronary vessels.

The differential in vivo effects of full-length VEGF-D versus VEGF-DΔNΔC indicate that alternatively processed or unprocessed forms of this growth factor could be useful in different clinical settings (Rissanen T. T. et al., Circ. Res. (92) 2003 1098-1106). For example, in certain scenarios, it may be beneficial to use a stable full-length form of VEGF-D to preferentially induce lymphangiogenesis whereas in other settings induction of both angiogenesis and lymphangiogenesis would be required. Therefore the availability of stable full-length or partially processed forms of VEGF-D would be highly desirable. This would require an approach for generating full-length or partially processed forms of VEGF-D that are unable to be further proteolytically processed in vivo. Furthermore, the availability of pure preparations of such derivatives would allow each to be biochemically and biologically characterized, in vitro and in vivo, in the absence of other derivatives arising from further processing. In contrast, some previous analyses of VEGF-D have utilized mixtures of different derivatives for in vitro and in vivo assays (Orlandini M. et al., Proc. Natl. Acad. Sci. USA (93) 1996 11675-11680).

Thus, there is a need for derivatives of VEGF-D that are resistant to proteolytic processing and that may be useful for various clinical applications.

### SUMMARY OF THE INVENTION

The present invention provides modified VEGF-D polypeptides that are resistant to proteolytic cleavage of one or both of the C-terminal propeptide and the N-terminal propeptide from the VEGF homology domain (VHD). Such peptides may comprise the C-terminal propeptide and the VHD, the N-terminal propeptide and the VHD, or the C-terminal propeptide, the VHD and the N-terminal propeptide.

In other embodiments, the present invention provides nucleic acids encoding the modified VEGF-D polypeptides, vectors comprising such nucleic acids, and cells comprising the vectors.

In another embodiment, the present invention is directed to methods of making the modified VEGF-D polypeptides. Compositions comprising the modified VEGF-D polypeptides are also provided.

The present invention also provides methods of using the modified VEGF-D polypeptides to affect angiogenesis, to affect lymphangiogenesis, and to inhibit stenosis and restenosis.

### BRIEF DESCRIPTION OF THE DRAWINGS

FIG. 1A shows the nucleotide sequence encoding human VEGF-D (SEQ ID NO: 1), and FIG. 1B shows the amino acid sequence of full-length human VEGF-D (SEQ ID NO: 2).

FIGS. 2A-C are diagrams schematically depicting the structure of full-length human VEGF-D, mutants thereof, the serine protease recognition sites, and the N- and C-terminal propeptides FIG. 2A shows that full-length human VEGF-D is cleaved by proprotein convertases that remove the N- and C-terminal propeptides (at the positions of the arrowheads) to generate mature VEGF-D. FIG. 2B depicts VEGF-D_{SSTS}, the point-mutated form from which the N-terminal propeptide (N) is not cleaved FIG. 3C depicts VEGF-D_{SSTS.IISS(1)} and VEGF-D_{SSTS.IISS(2)}: Point-mutated form from which the N- and C-terminal propeptides are not cleaved. (VEGF-D=human full-length VEGF-D, VEGF-D_{SSTS}=human full-length VEGF-D with R85S and R88S point mutations, VEGF-D_{SSTS.IISS}=human full-length VEGF-D with R85S, R88S and R204,205S point mutations.)

FIG. 3 is a Western blot showing that point mutations at the proteolytic recognition sites of human full-length VEGF-D completely inhibit proteolytic processing, whereas the wild-type full-length human VEGF-D is processed to various derivatives including the mature VHD. 293EBNA cells were transfected with point-mutated forms of human full-length VEGF-D. (N=N-terminal propeptide, C=C-terminal propeptide, VHD=VEGF homology domain). *fully mature recombinant VEGF-D, size is larger than mature VEGF-D cleaved from full-length material because of FLAG-tag (ca. 23 kDa)

FIGS. 4A-C show the sequences of human VEGF-D (4A) and truncated mutants (4B, C). The first residue of VEGF-D SSTS.ΔC derived from the VEGF-D sequence is shaded and double-underlined as is the first residue of VEGF-D ΔN.IISS derived from the VEGF homology domain of VEGF-D. These double-underlined residues are preceded by the residues “TR”, the FLAG octapeptide tag (“DYKDDDDK”; shaded) and the interleukin-3 signal sequence for protein secretion followed by the residues “ARQ” ( [symbol] ). The residues in the mutants that have been mutated to block proteolytic processing are bold and shaded (but not underlined). FIG. 4A is the amino acid sequence of wild-type full-length human VEGF-D. FIG. 4B is the amino acid sequence of VEGF-D SSTS.ΔC. FIG. 4C is the amino acid sequence of VEGF-D ΔN.IISS.

FIG. 5 is a schematic representation of full-length VEGF-D (A), VEGF-D SSTS.ΔC (B) and VEGF-D ΔN.IISS(C). The interleukin-3 signal sequence and FLAG tag are not depicted. “N” denotes the N-terminal propeptide. (VEGF-D=human full-length VEGF-D, VEGF-D_{SSTS}.ΔC=human VEGF-D with R85S and R88S point mutations and lacking C-terminal propeptide, VEGF-D ΔN.IISS=human VEGF-D with R204,205S point mutations and lacking the N-terminal propeptide).

FIG. 6 is a Western blot showing analysis of processing of human full-length wild-type VEGF-D (Full-length hVEGF-D), human VEGF-D SSTS.IISS (hVEGF-D SSTS.IISS), human VEGF-D SSTS.ΔC (hVEGF-D SSTS.dC) and human VEGF-D ΔN.IISS (hVEGF-D dN.IISS). Proteins were expressed in 293EBNA cells, immunoprecipitated and subjected to Western blot. The result from cells transfected with vector lacking sequence encoding VEGF-D (Empty vector) is included as negative control. The Western blot shows that point-mutated deletion mutants of VEGF-D do not undergo processing when transiently transfected in to 293EBNA1 human embryonic kidney cells (N=N-terminal propeptide, C=C-terminal propeptide, VHD=VEGF homology domain).

FIGS. 7A and B are graphs showing performance of mature VEGF-D (ΔNΔC), VEGF-D ΔN.IISS (ΔN) and the full-length mutant VEGF-D SSTS.IISS (FN) in bioassays of binding and cross-linking of the extracellular domains of VEGFR-2 (7A) and VEGFR-3 (7B).

### DESCRIPTION OF THE INVENTION

The present invention relates to variants or mutants of a VEGF-D molecule that are substantially resistant to proteolytic removal of the N- and/or C-terminal propeptides. Specifically, certain modifications of serine protease cleavage recognition sites render the VEGF-D polypeptides resistant to cleavage by serine proteases.

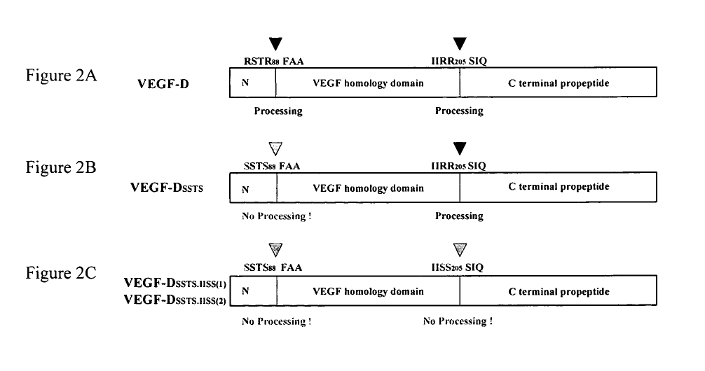

Sites of proteolysis in VEGF-D are known in the art. See, e.g., Stacker et al. J. Biol. Chem., 274 (1999) pp. 32127-32136. FIG. 2 schematically depicts two cleavage sites. One is a site of cleavage of the central VHD from the N-terminal propeptide, and the other is a site of cleavage of central VHD from the C-terminal propeptide. Modification of residues near these proteolysis sites in VEGF-D provide full-length VEGF-D that is stable and resistant to proteolytic processing, at one or both of these sites, by serine proteases.

Serine proteases recognize a site of RX(K/R)R or a site of RXₙR where n=0, 2, 4, or 6. The full-length VEGF-D contains multiple such recognition sites and any modifications of these sites that abolishes the ability to be cleaved by a serine protease is within the scope of this invention. It has been discovered in accordance with the present invention that modification of at least two of the R residues or insertion of 1, 3, or 5 amino acid residues between the two arginine residues of the RXₙR site, is sufficient to provide resistance to proteolytic processing by a serine protease. In other words, the site RX(K/R)R may be changed to BX(K/R)B, or RXJB, or BXJB, and the site RXₙR may be changed to BXₙB, where B is any amino acid other that R, and J is any amino acid other than R or K; and the site RXₙR, may be changed so that n=1, 3, or 5. It is to be understood that more than two changes, for example, the site RXₙR changed to BX₃B, also results in resistance to proteolytic processing.

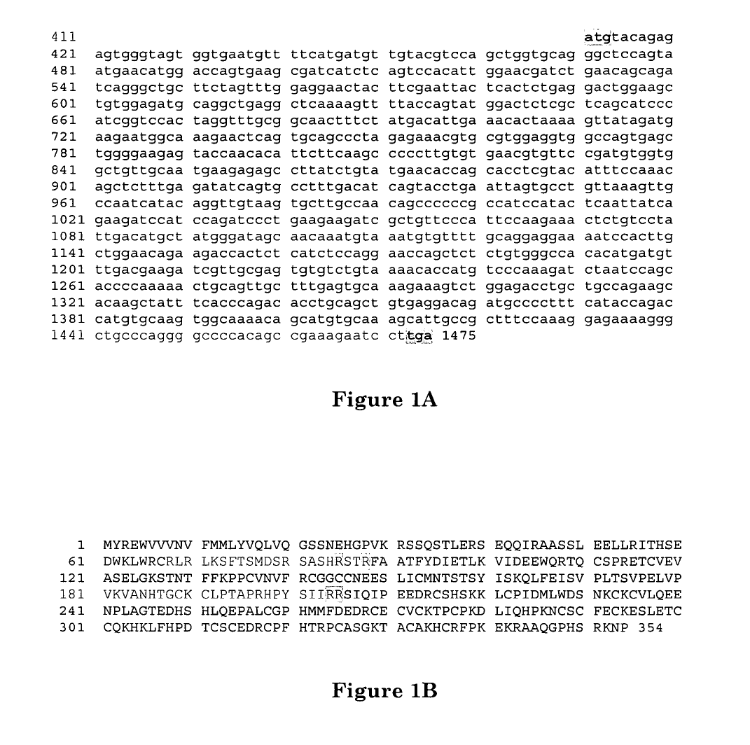

In one embodiment, the serine protease cleavage site, RSTR₈₈, at which the N-terminal propeptide of VEGF-D is cleaved from the VHD is modified. When used in the context of the present disclosure, the notation R₈₈ denotes the arginine residue at position 88 of the full-length VEGF-D polypeptide, as depicted in FIG. 1, and means that the amino acid residues at positions 85, 86, 87 and 88 are R, S, T and R, respectively. The notation “R88S” means that the amino acid residue at position 88 is changed from R(arginine) to S (serine).

In another embodiment, the serine protease cleavage site, IIRR₂₀₅, at which the C-terminal propeptide is cleaved from the VHD is altered. The notation IIRR₂₀₅ depicts the four amino acid residues at positions 202, 203, 204 and 205 of the VEGF-D full-length polypeptide as depicted in FIG. 1.

An ordinarily skilled artisan will readily recognize that any modification at these recognition sites that renders the polypeptide resistant to proteolytic processing by a serine protease is within the scope of the present invention. In a preferred embodiment, the two arginine residues at each of the above two serine protease cleavage sites are changed to any amino acid other than arginine. This change is sufficient to render the polypeptide resistant to proteolytic processing by a serine protease at the site.

A skilled artisan will also readily recognize that because arginine is a polar, positively charged amino acid, a polar, uncharged amino acid in its place would be suitable for achieving resistance to proteolytic processing. Thus, in one preferred embodiment, the two arginine residues of each of the serine protease recognition sites are changed to a cysteine, a serine, a threonine or tyrosine. Preferably, the amino acid residue substituting arginine is serine.

In order to minimize the impact of the modifications on the structure and function of the modified VEGF-D polypeptides, another polar charged amino acid, such as an aspartic acid, a glutamic acid, or a lysine, may be used to substitute the arginine residue. Similarly, histidine, also positively charged, may be used.

The capacity of the modifications discovered herein to block cleavage of the N-terminal propeptide from the VHD was unexpected because cleavage of the propeptides can occur at multiple, clustered positions in the full-length VEGF-D protein. For example, cleavage of the N-terminal propeptide can occur immediately after position 88 (arginine) or 99 (leucine) when full-length wild-type human VEGF-D is expressed in 293EBNA cells (Stacker S. A. et al., J. Biol. Chem. 274 (1999) 32127-32136). Modifications of the RSTR₈₈, however, prevent processing at both of these known cleavage sites.

Furthermore, it has been discovered herein that a VEGF-D polypeptide with a single amino acid substitution, R88S, alone, is insufficient to prevent the cleavage of the N-terminal propeptide from the VHD. Similarly, a single amino acid substitution, R205S, or R204S alone, is insufficient to prevent cleavage of the C-terminal or N-terminal propeptide from the VHD. Furthermore a mutant with the same single aa substitution R204S combined with a double aa mutation R85S, R88S, although resistant to N-terminal proteolytic processing, is not resistant to C-terminal proteolytic cleavage.

In a preferred embodiment, the present invention provides a full-length VEGF-D polypeptide wherein neither of the amino acids at positions corresponding to positions 85 and 88 of SEQ ID NO:2 is arginine. In another preferred embodiment, the present invention provides a full-length VEGF-D polypeptide wherein both of the amino acids at the positions corresponding to positions 85 and 88 of SEQ ID NO:2 are serine.

In another preferred embodiment, the present invention provides a full-length VEGF-D polypeptide wherein neither of the amino acids at positions corresponding to positions 204 and 205 of SEQ ID NO:2 is arginine. In another preferred embodiment, the present invention provides a full-length VEGF-D polypeptide wherein both of the amino acids at positions corresponding to positions 204 and 205 of SEQ ID NO:2 are serine.

In yet another preferred embodiment, the present invention provides a full-length VEGF-D polypeptide wherein none of the amino acids at positions corresponding to positions 85, 88, 204 and 205 of SEQ ID NO:2 is arginine. In another preferred embodiment, the present invention provides a full-length VEGF-D polypeptide wherein each of the amino acids at positions corresponding to positions 85, 88, 204 and 205 of SEQ ID NO:2 is serine.

In addition to full-length VEGF-D resistant to proteolytic processing, the present invention also relates to other stable derivatives of VEGF-D that are not proteolytically processed, e.g. a form consisting of the VHD and the C-terminal propeptide, or VHD and the N-terminal propeptide, as well as methods for generating the same. In a preferred embodiment, the polypeptide comprises the N-terminal propeptide and the VHD wherein neither of amino acids corresponding to positions 85 and 88 of SEQ ID NO:2 is arginine. In another preferred embodiment of such a polypeptide, each of the amino acids corresponding to positions 85 and 88 of SEQ ID NO:2 is serine. In another preferred embodiment, the polypeptide has the sequence of amino acids 1-205 of SEQ ID NO:2 wherein each of the amino acids at positions 85 and 88 is serine. In another preferred embodiment, the polypeptide has the sequence of amino acids 24-205 of SEQ ID NO:2 wherein each of the amino acids at positions 85 and 88 is serine.

In another preferred embodiment, the polypeptide comprises the VHD and the C-terminal propeptide wherein neither of the amino acids corresponding to positions 204 and 205 of SEQ ID NO:2 is arginine. In another preferred embodiment of such a polypeptide, each of the amino acids corresponding to positions 204 and 205 of SEQ ID NO:2 is serine. In another preferred embodiment, the polypeptide has the sequence of amino acids 206-354 of SEQ ID NO:2 wherein each of the amino acids at positions 204 and 205 is serine.

The polypeptides of the present invention are defined with reference to substantial identity to the amino acid sequence of SEQ ID NO:2. In particular, as used herein, the terms “native VEGF-D” and “full-length VEGF-D” refer to polypeptides having an amino acid sequence that has substantial identity to the amino acid sequence of SEQ ID NO:2. The term “N-terminal propeptide” refers to a polypeptide having an amino acid sequence that has substantial identity to amino acids 1 through 88 of SEQ ID NO:2. The term “C-terminal propeptide” refers to a polypeptide having an amino acid sequence that has substantial identity to amino acids 206 through 354 of SEQ ID NO:2. The term “VHD” refers to a polypeptide having an amino acid sequence that has substantial identity to amino acids 89 through 205 of SEQ ID NO:2. The term “substantial identity” means that two polypeptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity).

Optimal alignment of sequences for aligning a comparison window may be conducted by the local homology algorithm of Smith and Waterman (Smith and Waterman, Adv. Appl. Math. 2:482 (1981)), by the homology alignment algorithm of Needleman and Wunsch (Needleman and Wunsch, J. Mol. Biol. 48:443 (1970)), or by the search for similarity method of Pearson and Lipman (Pearson and Lipman, Proc. Natl. Acad. Sci. (U.S.A.) 85:2444 (1988), or by computerized implementations of these algorithms (GAP, BESTFIT, FASTA, and TFASTA in the Wisconsin Genetics Software Package Release 7.0, Genetics Computer Group, 575 Science Dr., Madison, Wis.).

The polypeptides may also be fragments, i.e., may have deletions of amino acids at the N-terminus and/or C-terminus. A deletion of about 20 to about 25 amino acids from the N-terminus of SEQ ID NO:2 is specifically contemplated.

The polypeptides may also comprise non-VEGF-D sequences, for example, to facilitate signaling, purification and identification. For example, the polypeptides of the present invention may contain the FLAG polypeptide sequence (DYKDDDDK). In another example, the polypeptide may contain the interleukin-3 signal sequence.

The present invention also relates to methods for producing the variant VEGF-D polypeptides described above. The polypeptides may be produced by recombinant methods or by direct chemical synthesis. Preferably, the method according to the present invention is based on site-directed mutagenesis to modify a polynucleotide molecule encoding VEGF-D.

Methods and technology for site-directed mutagenesis are well-established and well-known in the art. See for example, Braman (Ed.), In Vitro Mutagenesis Protocols, Methods in Molecular Biology, Vol. 182, 2nd Ed, Humana Press, Totowa, 2002, the entire contents and disclosure of which is hereby incorporated by reference.

In site-directed mutagenesis, one or more nucleotides at a specific site of the polynucleotide molecule encoding a peptide is altered with precision, such that the corresponding genetic code is changed and the amino acid residue(s) encoded is altered accordingly. A variety of very efficient methods are known, including both polymerase chain reaction (PCR)-based and non-PCR-based. Several convenient commercial kits are available. See Kunkel, Rapid and efficient site-specific mutagenesis without phenotype selection, Proc. Natl. Acad. Sci. USA, 82 (1985) pp. 488-492; Weiner et al., Site-directed mutagenesis of double-stranded DNA by the polymerase chain reaction, Gene, 151 (1994) pp. 119-123; Ishii et al., Site-directed mutagenesis, Methods Enzymol., 293 (1998) pp. 53-71, Sawano and A. Miyawaki, Directed evolution of green fluorescent protein by a new versatile PCR strategy for site-directed and semi-random mutagenesis, Nucleic Acids Research, Vol. 28, No. 16 (2000) p. e78, the entire contents and disclosure of which are hereby incorporated by reference.

Several multiple site-directed mutagenesis methods have also been described, using multiple rounds of PCR, including successive rounds of overlap extension PCR or successive rounds of PCR combined with in vitro dam-methylation/ligation before and DpnI digest and gel purification after each PCR step, see Michaelian et al. A general and fast method to generate multiple site directed mutations, Nucleic Acids Res., 20 (1992) p. 376; Dwivedi et al., Generation of multiple mutations in the same sequence via the polymerase chain reaction using a single selection primer, Anal. Biochem., 221 (1994) pp. 425-428; Bhat, Multiple site-directed mutagenesis, Methods Mol. Biol., 57 (1996) pp. 269-277; Meetei et al., Generation of multiple site-specific mutations in a single polymerase chain reaction product, Anal. Biochem., 264 (1998) pp. 288-291; Kim et al., Multiple site mutagenesis with high targeting efficiency in one cloning step, Biotechniques, 28 (2000) pp. 196-198; Lee et al., Multiple mutagenesis of non-universal serine codons of the Candida rugosa LIP2 gene and biochemical characterization of purified recombinant LIP2 lipase overexpressed in Pichia pastoris, Biochem. J., 366 (2002) pp. 603-611; and international patent application numbers WO 03/002761A1 and WO 99/25871. U.S. Pat. No. 6,878,531 also discloses a method for simultaneously producing multiple sites of mutagenesis.

The present invention further provides isolated nucleic acids that encode the VEGF-D polypeptides of the invention. In a preferred embodiment, the nucleic acids are linked to one or more regulatory elements that control or modulate the expression of the polynucleotides. The nucleic acid encoding the protease-resistant VEGF-D polypeptides of the present invention are preferably ligated into expression vectors, which further comprise regulatory elements including a transcriptional promoter and transcriptional terminator.

The present invention further provides vectors for delivery and expression of the nucleic acids of the invention. Such vectors are known in the art and include, for example, chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, adenoassociated viruses, densoviruses, poxviruses, pseudorabies viruses, and retroviruses.

In a preferred embodiment, the vector comprises a constitutive or inducible promoter operably linked to a nucleic acid encoding the VEGF-D polypeptide. Inducible promoters are well known and readily available to those of skill in the art, and include for example the tet-off and tet-on vectors from BD Biosciences (San Jose, Calif.). Hybrid promoters may also be used to improve inducible regulation of the expression construct. The promoter can additionally include features to ensure or to increase expression in a suitable host. The use of tissue-specific promoters or enhancers may be desirable to limit expression of the polynucleotide to a particular tissue type.

Constitutive promoters are also well known and readily available, and include for example the CMV promoter. A constitutive promoter may be selected to direct the expression of the desired polypeptide of the present invention. Suitable promoters include, for example, LTR, SV40 and CMV in mammalian systems; E. coli lac or trp in bacterial systems; baculovirus polyhedron promoter (polh) in insect systems and other promoters that are known to control expression in eukaryotic and prokaryotic cells. Examples of strong constitutive and/or inducible promoters which are preferred for use in fungal expression hosts are those which are obtainable from the fungal genes for xylanase (xlnA), phytase, ATP-synthetase, subunit 9 (oliC), triose phosphate isomerase (tpi), alcohol dehydrogenase (AdhA), α-amylase (amy), amyloglucosidase (AG—from the glaA gene), acetamidase (amdS) and glyceraldehyde-3-phosphate dehydrogenase (gpd) promoters. Examples of strong yeast promoters are those obtainable from the genes for alcohol dehydrogenase, lactase, 3-phosphoglycerate kinase and triosephosphate isomerase. Examples of strong bacterial promoters include SP02 promoters as well as promoters from extracellular protease genes.

The expression vector may also contain sequences which act on the promoter to amplify expression. For example, the SV40, CMV, and polyoma cis-acting elements (enhancer) and a selectable marker can provide a phenotypic trait for selection (e.g. dihydrofolate reductase or neomycin resistance for mammalian cells or amplicillin/tetracyclin resistance for E. coli). Selection of the appropriate vector containing the appropriate promoter and selection marker is well within the level of those skilled in the art.

The present invention also provides compositions comprising the variant VEGF-D polypeptides of the invention, or nucleic acids of the invention encoding the VEGF-D polypeptides, as well as methods of using the compositions. In a preferred embodiment, the composition comprises an expression vector comprising the nucleic acid encoding the variant VEGF-D polypeptide. More preferably, the expression vector is a plasmid, or a viral vector.

The vectors may be delivered to a cell of a patient, in vivo or ex vivo, by transformation or transfection of a cell via a vector, including viral vectors such as vectors based on adenovirus, adeno-associated virus, lentivirus, retrovirus or derivatives thereof, and plasmids, to result in expression of the variant VEGF-D of the present invention in the cell. In one embodiment, stable transfection and constitutive expression of vectors is achieved, or such expression may be under the control of tissue or development- or tissue-specific or inducible promoters. Delivery can also be achieved by liposomes. For in vivo therapy, direct delivery of polynucleotides may be used instead of stable transfection of an expression vector.

When used clinically, the vectors of the present invention may be delivered intradermally, intramuscularly (for example in the skeletal muscle or cardiac muscle), into the vessel wall of coronary arteries via balloon angioplasty delivery, intraocularly, or intravascularly (including intraarterially). In preferred embodiments, delivery may be a combination of two or more of the various delivery methods.

The present invention further relates to cells that comprise the polynucleotide of the present invention, preferably in the form of expression vectors. Many choices of cell lines, preferably mammalian, more preferably human, cell lines, are suitable as the host cell for the present invention.

The protease-resistant VEGF-D polypeptides of the present invention that activate VEGFR-2 are useful in methods of inducing angiogenesis. Accordingly, the present invention provides a method of inducing angiogenesis in a mammal in need thereof comprising administering a VEGF-D polypeptide of the present invention to the mammal in an amount effective to induce angiogenesis. Such methods are useful for the treatment of ischemic heart disease and peripheral arterial disease.

The protease-resistant VEGF-D polypeptides of the present invention that activate VEGFR-3 are useful in methods of inducing lymphangiogenesis. Accordingly, the present invention provides a method of inducing lymphangiogenesis in a mammal in need thereof comprising administering a VEGF-D polypeptide of the present invention to the mammal in an amount effective to induce lymphangiogenesis. Such methods are useful for the treatment of primary and secondary lymphedema.

The protease-resistant VEGF-D polypeptides of the present invention are also useful in methods of inhibiting stenosis or restenosis. Stenosis of blood vessels is a complication of vascular surgery. Stenosis also occurs in tubes and stents used as access grafts for hemodialysis. Restenosis is the re-narrowing of an artery after it has been treated with angioplasty or stenting. The polypeptides of the invention may be administered to a patient in need of inhibition of stenosis or restenosis. In another embodiment, stents coated with the polypeptides of the invention, or configured to elute the polypeptides when the stent is used in a subject, are also provided by the present invention. One of ordinary skill in the art can produce such stents by methods known in the art and disclosed, for example, in U.S. Pat. Nos. 7,048,962 and 6,702,850. The polypeptides of the invention may be provided in the form of vectors comprising nucleic acids that encode the polypeptides.

In the foregoing methods, the VEGF-D polypeptides may be administered as a composition comprising the polypeptide or a vector comprising a nucleic acid that encodes the polypeptide as described hereinabove.

The polypeptides of the present invention may be administered as pharmaceutical compositions comprising the polypeptides and a pharmaceutically acceptable carrier. The formulation of pharmaceutical compositions is generally known in the art and reference can conveniently be made to Remington's Pharmaceutical Sciences, 17th ed., Mack Publishing Co., Easton, Pa. Formulations of the polypeptides for use in present invention are stable under the conditions of manufacture and storage and are preserved against the contaminating action of microorganisms such as bacteria and fungi. Prevention against microorganism contamination can be achieved through the addition of various antibacterial and antifungal agents.

The polypeptides are compounded for convenient and effective administration in pharmaceutically effective amounts with a suitable pharmaceutically acceptable carrier and/or diluent in a therapeutically effective dose.

As used herein, the term “pharmaceutically acceptable carrier and/or diluent” includes any and all solvents, dispersion media, antibacterial and antifungal agents, microcapsules, liposomes, cationic lipid carriers, isotonic and absorption delaying agents and the like which are not incompatible with the active ingredients. The use of such media and agents for pharmaceutical active substances is well known in the art. Supplementary active ingredients may also be incorporated into the compositions and used in the methods of present invention.

The precise therapeutically effective amount of the polypeptides of the invention to be used in the methods of this invention applied to humans can be determined by the ordinary skilled artisan with consideration of individual differences in age, weight, extent of disease and condition of the patient.

In the methods according to the present invention, the polypeptide may be administered in a manner compatible with the dosage formulation, in such amount as will be therapeutically effective, and in any way which is medically acceptable. Possible administration routes include injections by parenteral routes such as intravascular, intravenous, intraarterial, subcutaneous, intramuscular, intratumor, intraperitoneal, intraventricular or intraepidural. The compositions may also be directly applied to tissue surfaces, for example, during surgery. Sustained release administration is also specifically included in the invention, by such means as depot injections or erodible implants.

The protease-resistant VEGF-D polypeptides of the invention that bind to but do not activate VEGFR-2 and/or VEGFR-3 are useful in methods of inhibiting the biological activity of endogenous VEGF-D, VEGF-C and/or VEGF-A by blocking receptor access to these growth factors. Such polypeptides are thus useful for the treatment of excessive angiogenesis and/or lymphangiogenesis, e.g. in the treatment of cancer.

In another embodiment of the present invention, the biological activity of VEGF-D is inhibited by delivering a protease-resistant VEGF-D polypeptide of the present invention to a tumor cell, preferably by delivery in a viral vector comprising a nucleic acid that encodes the polypeptide. Dimerization of the polypeptide with endogenous VEGF-D or VEGF-C may result in hybrid molecules with reduced capacity for activation by processing relative to endogenous growth factor dimers.

The following non-limiting examples serve to further illustrate the present invention. It is to be understood that these examples serve only to describe the specific embodiments of the present invention, but do not in any way limit the scope of the claims.

### EXAMPLES

### Example 1

### Double Mutations R85S and R88S Completely Abolished Furin Cleavage of the N-Terminal Propeptide, and Double Mutations R204S and R205S Completely Abolished Furin Cleavage of the C-Terminal Propeptide Cleavage Site

1. Materials and Methods

The plasmid pEFsBOS-FullNFlag encoding full-length human VEGF-D with a NH₂-terminal FLAG-octapeptide tag (referred to henceforth as pEFsBOS-VEGF-D; Stacker S. A. et al., J. Biol. Chem. 274 (1999) 32127-32136) was used for point mutation by polymerase chain reaction (PCR) with the Quikchange Site-Directed Mutagenesis Kit (Stratagene, CA, USA). The aim was to generate a cDNA encoding full-length VEGF-D with the following four specific mutations: R85S, R88S, R204S & R205S.

R85S and R88S Mutations.

The amino acid sequence immediately before the site at which the N-terminal propeptide of VEGF-D is cleaved from the VHD is RSTR₈₈ (encoded by CGG-TCC-ACT-AGG). A compound mutant containing the conversions R85S and R88S was generated by the following steps and designated SSTS₈₈ (encoded by TCC-TCC-ACT-TCC):

1) For the R85S mutation pEFsBOS-VEGF-D was amplified with the following primers: forward 5′-GCA TCC CAT CGG TCC ACT TCC TTT GCG GCA ACT TTC TAT G-3′; reverse 5′-CAT AGA AAG TTG CCG CAA AGG AAG TGG ACC GAT GGG ATG C-3′. The resulting vector is designated pEFsBOS-VEGF-D_{RSTS}.

2) For the R88S mutation pEFsBOS-VEGF-D_{RSTS} was amplified with the following primers: forward 5′-CTC GCT CAG CAT CCC ATT CCT CCA CTT CCT TTG CGG-3′; reverse 5′-CCG CAA AGG AAG TGG AGG AAT GGG ATG CTG AGC GAG-3′. The resulting vector is designated pEFsBOS-VEGF-D_{SSTS}.

R204S and R205S Mutations.

The amino acid sequence immediately before the site at which the C-terminal propeptide is cleaved from the VHD is IIRR₂₀₅ (encoded by ATT-ATC-AGA-AGA). This was converted to IISS₂₀₅ (encoded by ATT-ATC-AGC-AGC or ATT-ATC-TCC-AGC). pEFsBOS-VEGF-D_{SSTS} was used as template and the following primers were used:

1) For the R204 and R205S mutations pEFsBOS-VEGF-D_{SSTS} was amplified with the following primers: forward 5′-CCA TAC TCA ATT ATC AGC AGC TCC ATC CAG ATC CCT GAA G-3′; reverse 5′-CTT CAG GGA TCT GGA TGG AGC TGC TGA TAA TTG AGT ATG G-3′. The resulting vector is designated pEFsBOS-VEGF-D_{SSTS.IISS(1)}.

2) A second approach was also used to generate the R204 and R205S mutations in which pEFsBOS-VEGF-D_{SSTS} was amplified with the following primers: forward 5′-CCA TAC TCA ATT ATC TCC AGC TCC ATC CAG ATC CCT GAA G-3′; reverse 5′-CTT CAG GGA TCT GGA TGG AGC TGG AGA TAA TTG AGT ATG G-3′. The resulting vector is designated pEFsBOS-VEGF-D_{SSTS.IISS(2)}. The encoded amino acid sequence for VEGF-D in this plasmid is the same as for pEFsBOS-VEGF-D_{SSTS.IISS(1)}. These plasmids differ only in the codon encoding the mutated serine residue at position 204.

The PCR programs were according to the manufacturer and the resulting plasmids were transformed into E. coli and purified using DNA plasmid purification kits (Qiagen, Hilden, Germany). All the resulting point-mutated VEGF-D coding regions were fully sequenced. Subsequently, the plasmids were used to transfect 293EBNA human embryonic kidney cells. These cells were grown in Dulbecco's Modified Eagle's Medium supplemented with 10% fetal calf serum, penicillin, streptomycin and L-glutamine. The cells were transiently transfected using FuGENE 6 Transfection Reagent (Roche Diagnostics, Basel, Switzerland) according to the manufacturer's protocol with the parent expression vector lacking sequence encoding VEGF-D (pEFsBOS), a transfection control plasmid encoding Green Fluorescent Protein (pEF-GFP), or with plasmids encoding full-length VEGF-D (pEFsBOS-VEGF-D), mature VEGF-D (pEFsBOS-VEGF-DΔNΔC) or the mutant forms of full-length human VEGF-D (pEFsBOS-VEGF-D_{SSTS}, pEFsBOS-VEGF-D_{SSTS.IISS(1)} and pEFsBOS-VEGF-D_{SSTS.IISS(2)}). Conditioned media were collected after 48 hours and subjected to immunoprecipitation using polyclonal rabbit anti-human VEGF-D antiserum (A2) that binds the VHD of VEGF-D, and Protein-A-Sepharose as described previously (Stacker S. A. et al., J. Biol. Chem. 274 (1999) 32127-32136). The samples were subjected to electrophoresis in a reduced NuPage 4-12% gradient gel (Invitrogen, CA, USA) and transferred to a nitrocellulose filter. Immunoblotting was performed with biotinylated anti-human VEGF-D antibodies (R&D Systems, MN, USA) and filters were subjected to electrogenerated chemoluminescence (ECL) reaction and exposed to film.

2. Results

In an attempt to generate forms of full-length human VEGF-D that would not be subject to proteolytic processing, amino acid residues near the sites of cleavage of the central VHD from the N-terminal propeptide (arginine residues at positions and 85 and 88) and from the C-terminal propeptide (arginine residues at positions 204 and 205) were mutated to serine residues. Two plasmid constructs were generated in which all four of these residues were mutated to serine, designated pEFsBO S-VEGF-D_{SSTS.IISS(1)} and pEFsBOS-VEGF-D_{SSTS.IISS(2)}. These plasmids encode an identical VEGF-D amino acid sequence but differ in the sequence of the codon encoding the mutated serine residue at position 204. Both plasmids were used in case the alternative codon usage effected the expression of the encoded VEGF-D protein. In addition, a plasmid encoding full-length VEGF-D mutated to serine residues only at positions 85 and 88 (pEFsBOS-VEGF-D_{SSTS}) was employed. These plasmids were used to transiently transfect 293EBNA cells, along with control plasmids encoding wild-type full-length human VEGF-D and mature VEGF-D, to assess the capacity of the encoded VEGF-D proteins to be proteolytically processed. These cells were chosen for the analysis as they promote cleavage of both propeptides from the VHD (Stacker S. A. et al., J. Biol. Chem. 274 (1999) 32127-32136).

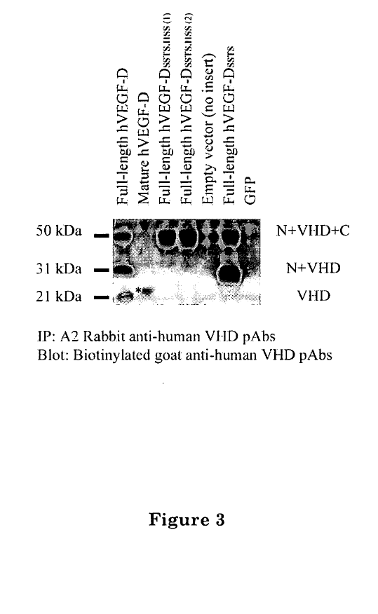

VEGF-D proteins expressed by transiently transfected 293EBNA cells were analyzed by immoprecipitation followed by Western blotting (FIG. 3). When wild-type full-length VEGF-D was expressed in these cells, three derivatives were detected: full-length VEGF-D (˜50 kDa), a partially processed form containing the N-terminal propeptide and the VHD (˜31 kDa) and the mature form (˜21 kDa), as reported previously (Stacker S. A. et al., J. Biol. Chem. 274 (1999) 32127-32136). In contrast, the full-length VEGF-D mutant protein encoded by pEFsBOS-VEGF-D_{SSTS.IISS(1)} or pEFsBOS-VEGF-D_{SSTS.IISS(2)} was not proteolytically processed at all as only the full-length form (˜50 kDa) could be detected. In contrast, the C-terminal propeptide of the mutant form VEGF-D_{SSTS} could be cleaved from the protein to generate a partially processed derivative containing the N-terminal propeptide and VHD (˜31 kDa) but the N-terminal propeptide was not removed as no mature form could be detected, nor any of a form containing only the VHD and C-terminal propeptide (predicted molecular mass of ˜40 kDa). These findings demonstrate that the VEGF-D_{SSTS.IISS} mutant form of full-length human VEGF-D is resistant to removal of both propeptides whereas VEGF-D_{SSTS} is resistant to removal of the N-terminal propeptide only.

### Example 2

### Single Substitution R88S Alone is not Sufficient to Abolish Serine Protease Recognition of the Cleavage Site

A VEGF-D mutant with a single amino acid substitution, R88S, was generated using the methods described above, and was designated VEGF-D_{RSTS.IIRR}. The substitution is located immediately adjacent to where the N-terminal propeptide is cleaved from the VHD. Expression in 293 cells and IP/Western analysis (as described above) revealed that the N-terminal and C-terminal propeptides were cleaved from the VHD of this mutant protein, as for the wild-type VEGF-D. This shows that mutation R88S alone is not capable of preventing cleavage of the N-terminal or C-terminal propeptides from the VHD.

### Example 3

### Single Substitution R205S Alone is not Sufficient to Abolish Serine Protease Recognition of the Cleavage Site

A VEGF-D mutant with a single amino acid substitution, R205S was also generated and designated VEGF-D_{RSTR.IIRS}. The substitution is immediately adjacent to where the C-terminal propeptide is cleaved from the VHD. Expression in 293 cells and IP/Western analysis revealed that the N-terminal and C-terminal propeptides were cleaved from the VHD of this mutant protein, as for wild-type VEGF-D. This shows that mutation R205S alone is not capable of preventing cleavage of the C-terminal or N-terminal propeptide from the VHD.

### Example 4

### Single Substitution R204S Alone Allowed Only Negligible Serine Protease Recognition of the Cleavage Site

A single mutant with C-terminal R204S substitution was generated and designated VEGF-D_{RSTR.IISR}. This mutant was negligibly proteolytically processed at the C-terminal and N-terminal processing sites in native conditions. Upon co-transfection with human furin the mutant was fully processed at the C-terminal (IISR) and N-terminal (RSTR) processing sites.

### Example 5

### A Single Substitution R204S at the C-Terminal Processing Site is not Sufficient to Abolish Serine Protease Recognition of the Cleavage Site Even when Combined with Double Mutation R85S and R88S

A mutant with the single amino acid substitution R204S combined with a double amino acid mutation R85S, R88S (which abolishes N-terminal proteolytic processing in the native state and in the presence of human Furin), was generated and designated VEGF-D_{SSTS.IISR}. This mutant was resistant to the N-terminal proteolytic processing, but remained able to be processed at the C-terminal processing site. This further confirmed that a single mutation R204S at the C-terminal processing site is not sufficient to abolish its recognition by furin.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. All references cited hereinabove and/or listed below are hereby expressly incorporated by reference.

### Example 6

### Generation of Full-Length VEGF-D, VEGF-DΔC and VEGF-DΔN that are Resistant to Proteolytic Processing

Materials and Methods

Generation of VEGF-D SSTS.ΔC

PCR was done using standard protocol with primers: fwd 5′-GCG TCT AGA CTA GTG CTA GC TCA TCA TCT TCT GAT AAT TGA GTA-3′, rev 5′-CCA GCT TGG CAC TTG ATG TA-3′ and using pEFsBOS-VEGF-D_{SSTS} (described hereinabove) as template. The resulting PCR fragment was digested with XbaI and inserted into a XbaI-digested pApex3 vector. The DNA encoding this protein construct, termed VEGF-D SSTS.ΔC, was verified using restriction enzyme digests and nucleotide sequencing.

Generation of VEGF-D ΔN.IISS

PCR was done according to standard protocol with primers: fwd 5′-GCG ACG CGT TTT GCG GCA ACT TTC TAT-3′, rev 5′-GAT GGG GAA CAC TGC TGT TTA and using pEFsBOS-VEGF-D_{SSTS.IISS} as template. The resulting PCR fragment was digested with MluI and inserted into MluI-digested pEFsBOS vector. This construct was subsequently digested with XbaI and the fragment encoding the VEGF-D mutant was ligated into a XbaI-digested pApex3 vector. The DNA encoding this protein construct, termed VEGF-D ΔN.IISS, was verified using restriction enzyme digests and nucleotide sequencing.

Protein Expression and Western Blotting

Plasmids were used to transfect 293EBNA human embryonic kidney cells. These cells were grown in Dulbecco's Modified Eagle's Medium supplemented with 10% fetal calf serum, penicillin, streptomycin and L-glutamine. The cells were transiently transfected using Lipofectamine 2000 transfection reagent (Invitrogen, CA, USA) according to the manufacturer's protocol with the parent expression vector lacking sequence encoding VEGF-D (pApex3), with plasmid encoding full-length VEGF-D (pApex3-VEGF-D), or the mutant forms of human VEGF-D: pApex3-VEGF-D SSTS.IISS, pApex3-VEGF-D SSTS.ΔC or pApex3-VEGF-D ΔN.IISS. Conditioned media were collected after 48 hours and subjected to immunoprecipitation using polyclonal rabbit anti-human VEGF-D antiserum (A2) that binds the VHD of VEGF-D, and Protein-A-Sepharose as described previously (Stacker et al., J. Biol. Chem. 274:32127-32136, 1999). The samples were subjected to electrophoresis in reduced NuPage 4-12% gradient gel (Invitrogen, CA, USA) and transferred to a nitrocellulose filter. Immunoblotting was performed with biotinylated anti-human VEGF-D antibodies that bind the VHD (R&D Systems, MN, USA) and filters were subjected to chemoluminescence (ECL) reaction and exposed to film.

Protein Purification and Bioassays of Receptor Binding and Cross-Linking

The VEGF-D variants were purified from conditioned media by affinity chromatography on M2 (anti-FLAG) gel and concentrated as described previously (Stacker et al., J. Biol. Chem. 274:32127-32136, 1999). The proteins were quantitated spectrophotometrically. Samples of protein were used in bioassays of binding and cross-linking of the extracellular domains of VEGFR-2 and VEGFR-3. These assays employ IL-3-dependent Ba/F3 pre-B cells expressing chimeric receptors consisting of the extracellular domains of mouse VEGFR-2 or human VEGFR-3 and the cytoplasmic domain of the erythropoietin receptor (Stacker et al., J. Biol. Chem. 274:32127-32136, 1999; Stacker et al., J. Biol. Chem. 274:34884-34892, 1999; Achen et al. Eur. J. Biochem. 267:2505-2515, 2000). In the absence of IL-3, binding and cross-linking of the chimeric receptors in these assays leads to cell survival and proliferation which was monitored by incorporation of tritiated-thymidine.

Two Novel VEGF-D Mutant Proteins

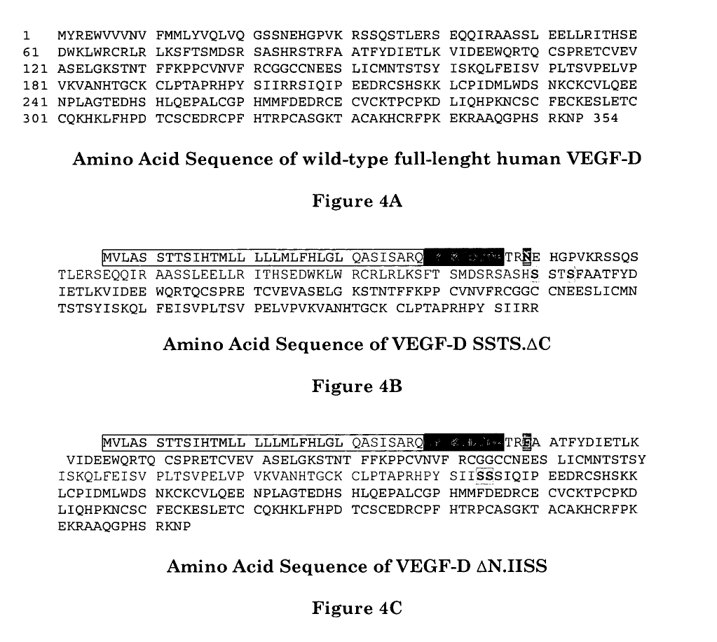

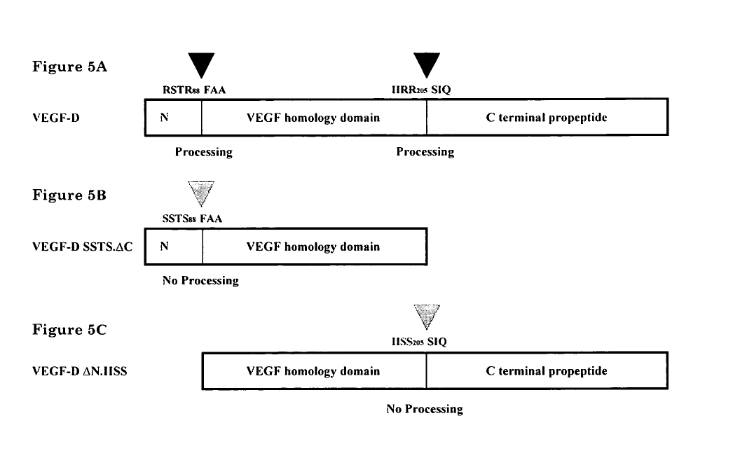

The VEGF-D SSTS.ΔC mutant lacks the C-terminal propeptide of full-length VEGF-D (see FIG. 4A for amino acid sequence of full-length VEGF-D and FIG. 5A for schematic representation). This mutant contains the region of human VEGF-D from amino acid 24 (asparagine) to 205 (arginine). The cleavage site RSTR₈₈↓ in the wild-type protein, at which the N-terminal propeptide is cleaved from the VEGF homology domain, is altered to SSTS in this mutant (see FIG. 4B for sequence of this mutant and FIG. 5B).

The VEGF-D ΔN.IISS mutant lacks the N-terminal propeptide of full-length VEGF-D. This mutant contains the region of VEGF-D from amino acid 89 (phenylalanine) to 354 (proline) of human VEGF-D (FIG. 4C and FIG. 5C). The cleavage site IIRR₂₀₅↓ in wild-type VEGF-D, at which the C-terminal propeptide is cleaved from the VEGF homology domain, is altered to IISS in this mutant.

Results

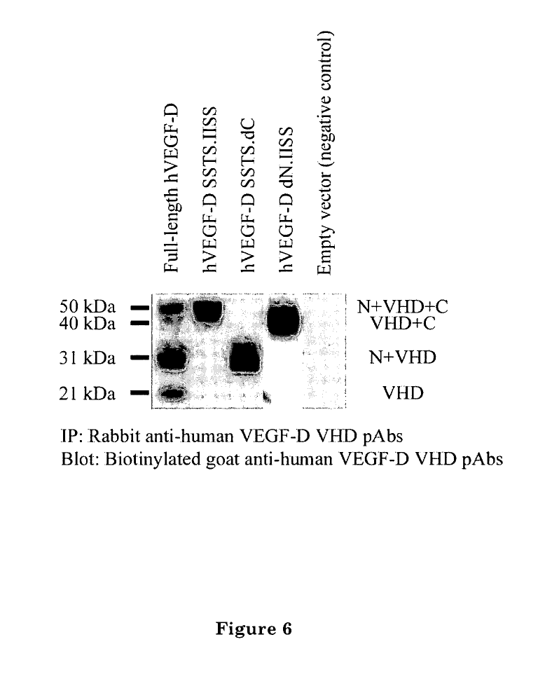

The mutants VEGF-D SSTS.ΔC and VEGF-D ΔN.IISS were tested for proteolytic processing alongside wild-type full-length VEGF-D and the full-length mutant VEGF-D SSTS.IISS. These proteins were expressed in 293EBNA cells by transient transfection with expression plasmids and analyzed by immunoprecipitation and Western blotting with an antibody that binds the VEGF homology domain, as described in Materials and Methods. As expected, full-length wild-type VEGF-D was partially processed to generate a form consisting of the N-terminal propeptide and the VEGF-homology domain (˜31 kDa) and the mature form consisting only of the VEGF homology domain (˜20 kDa) (Stacker et al., J. Biol. Chem. 274:32127-32136, 1999) (FIG. 6). In addition, some of the full-length form (˜50 kDa) was also detected. In contrast, VEGF-D SSTS.IISS was not processed at all—only the full-length form was detected. Likewise, VEGF-D SSTS.ΔC and VEGF-D ΔN.IISS were not processed to generate the mature form. This contrasts with previous studies, employing forms of VEGF-DΔN and VEGF-DΔC that were not mutated at processing sites, in which these derivatives were efficiently processed to mature VEGF-D (Stacker et al., J. Biol. Chem. 274:32127-32136, 1999; McColl et al., unpublished). These finding indicate that VEGF-D SSTS.IISS, VEGF-D SSTS.ΔC and VEGF-D ΔN.IISS are resistant to proteolytic processing in this cell-based assay system.

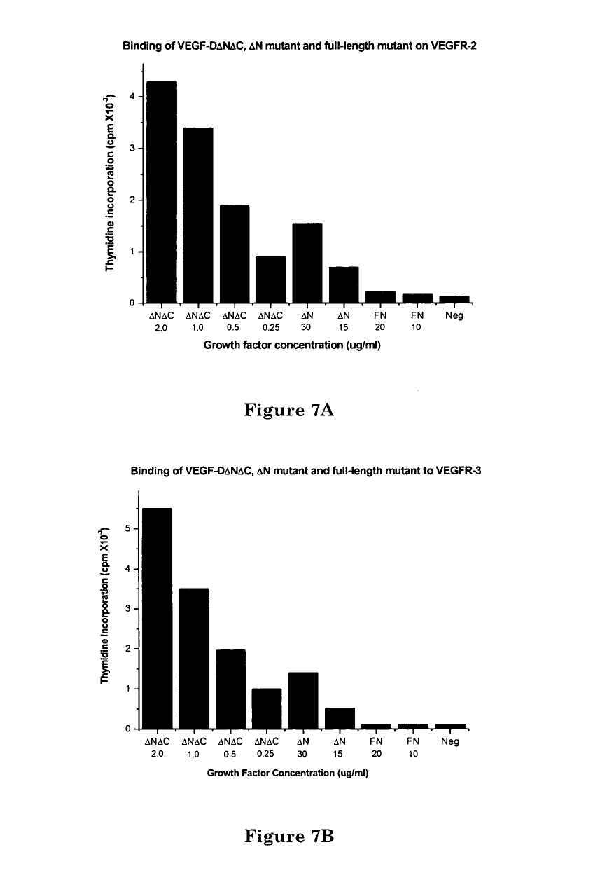

The capacity of VEGF-D mutants to bind and cross-link the VEGFR-2 and VEGFR-3 extracellular domains at the cell surface was tested in bioassays involving Ba/F3 cells expressing chimeric receptors containing these extracellular domains. As reported previously, the mature form of human VEGF-D (VEGF-DΔNΔC) was very active in both the VEGFR-2 and VEGFR-3 bioassays (FIG. 7) indicating that this protein effectively binds and cross-links the extracellular domains of these receptors (Achen et al., Proc. Natl. Acad. Sci. USA 95:548-553, 1998; Achen et al. Eur. J. Biochem. 267:2505-2515, 2000). The mutant VEGF-D ΔN.IISS also exhibited activity in both bioassays but was approximately 70-fold less active than mature VEGF-D in both cases. In contrast, activity of VEGF-D SSTS.IISS was only barely detectable in the VEGFR-2 assay and was undetectable in the VEGFR-3 assay. These results demonstrate that VEGF-D processing mutants exhibit distinct receptor-binding properties in comparison to mature VEGF-D.

What is claimed is:

1. A modified VEGF-D polypeptide that comprises a VEGF homology domain (VHD), and at least one of (1) a C-terminal propeptide that is resistant to cleavage from the VHD by a serine protease and (2) an N-terminal propeptide that is resistant to cleavage from the VHD by a serine protease.

2. The modified VEGF-D polypeptide according to claim 1, which comprises a C-terminal propeptide resistant to cleavage from the VHD by a serine protease.

3. The modified VEGF-D polypeptide according to claim 2, which further comprises an N-terminal propeptide resistant to cleavage from the VHD by a serine protease.

4. The modified VEGF-D polypeptide according to claim 1, which comprises an N-terminal propeptide resistant to cleavage from the VZHD by a serine protease.

5. The VEGF-D polypeptide according to claim 1, wherein the serine protease is selected from the group consisting of furin, PC5, PC7 and plasmin.

6. The VEGF-D polypeptide according to claim 5, wherein the serine protease is furin.

7. A VEGF-D polypeptide that comprises an amino acid sequence corresponding to SEQ ID NO: 2 wherein neither amino acid residue at position 85 or 88 is arginine, or neither amino acid residue at position 204 or 205 is arginine.

8. The VEGF-D polypeptide according to claim 7, wherein both amino acid residues at positions 85 and 88 are arginine.

9. The VEGF-D polypeptide according to claim 7, wherein both amino acid residues at positions 204 and 205 are arginine.

10. The VEGF-D polypeptide according to claim 7, wherein none of the amino acid residues at positions 85, 88, 204 and 205 is arginine.

11. The VEGF-D polypeptide according to claim 7, wherein at least one of the amino acid residues at positions 85, 88, 204 and 205 is serine.

12. The VEGF-D polypeptide according to claim 7, wherein at least two of the amino acid residues at positions 85, 88, 204 and 205 are serine.

13. The VEGF-D polypeptide according to claim 7, wherein at least three of the amino acid residues at positions 85, 88, 204 and 20 are serine.

14. The VEGF-D polypeptide according to claim 7, wherein each of the amino acid residues at positions 85, 88, 204 and 205 is serine.

15. A modified VEGF-D polypeptide that comprises an amino acid sequence corresponding to SEQ ID NO:2 wherein each of the amino acid residues at positions 85, 88, 204 and 205 is serine.

16. A polypeptide comprising the N-terminal propeptide and VHD of VEGF-D wherein neither amino acid residue at positions 85 and 88 is arginine.

17. The polypeptide of claim 16 wherein each of the amino acid residues at positions 85 and 88 is serine.

18. A polypeptide comprising the VHD and C-terminal propeptide of VEGF-D wherein neither amino acid residue at positions corresponding to positions 204 and 205 is arginine.

19. The polypeptide of claim 18 wherein each of the amino acids at positions corresponding to positions 204 and 205 is serine.

20. A nucleic acid molecule that encodes a modified VEGF-D polypeptide wherein said polypeptide comprises a VEGF homology domain (VHD), and at least one of (1) a C-terminal propeptide that is resistant to cleavage from the VHD by a serine protease and (2) an N-terminal propeptide that is resistant to cleaveage from the VHD by a serine.

21. A nucleic acid molecule that encodes a VEGF-D polypeptide that comprises an amino acid sequence corresponding to SEQ ID NO: 2 wherein neither amino acid residue at position 85 or 88 is arginine, or neither amino acid residue at position 204 or 205 is arginine.

22. A vector comprising a nucleic acid molecule that encodes a modified VEGF-D polypeptide wherein said polypeptide comprises a VEGF homology domain (VHD), and at least one of (1) a C-terminal propeptide that is resistant to cleavage from the VHD by a serine protease and (2) an N-terminal propeptide that is resistant to cleaveage from the VHD by a serine, wherein said nucleic acid is operably linked to a regulatory element.

23. The vector of claim 22, which is an expression vector.

24. The vector of claim 23, which is a prokaryotic expression vector or a eukaryotic expression vector.

25. The vector of claim 23, which is viral based, a plasmid, or a mammalian expression vector.

26. A cell comprising the vector of claim 23.

27. The cell according to claim 26, which is a mammalian cell.

28. The cell according to claim 27, which is a human cell.

29. A method for making the modified VEGF-D polypeptide of claim 1 comprising: preparing a polynucleotide encoding the modified polynucleotide via site-directed mutagenesis; and expressing said polynucleotide.

30. The method according to claim 29, wherein the site-directed mutagenesis is PCR-based.

31. A method for selectively/differentially activating VEGFR-2 or VEGFR-3 in a cell, the method comprising applying to the cell an effective amount of the modified VEGF-D polypeptide of claim 1.

32. The method according to claim 31, wherein VEGFR-2 is selectively activated, and wherein the modified VEGF-D polypeptide comprises a C-terminal propeptide that is not cleavable by a serine protease, and an N-terminal propeptide that is not cleavable by a serine protease.

33. A method for selectively/differentially activating VEGFR-2 or VEGFR-3 in a cell, the method comprising applying to the cell an effective amount of a nucleic acid of claim 20.

34. The method according to claim 33, wherein the nucleic acid is operatively linked to a regulatory element.

35. A method of inducing lymphangiogenesis in a mammal comprising administering a composition comprising the polypeptide of claim 1 to said mammal in an amount effective to induce lymphangiogenesis.

36. The method of claim 35 wherein said mammal is a human.

37. A method of inducing angiogenesis in a mammal comprising administering a composition comprising the polypeptide of claim 1 to said mammal in an amount effective to induce angiogenesis.

38. The method of claim 37 wherein said mammal is a human.

39. A method of inhibiting stenosis or restenosis in a mammal comprising administering a composition comprising the polypeptide of claim 1 to said mammal in an amount effective to inhibit stenosis or restenosis.

40. The method of claim 39 wherein said mammal is a human.

41. The method of claim 39 wherein the composition is administered as a stent comprising the polypeptide.

42. The method of claim 41 wherein the stent is configured to elute the polypeptide.

43. A composition comprising a modified VEGF-D polypeptide according to claim 1.

44. A composition comprising the vector according to claim 22.


SEQUENCE LISTING (24 sequences)

SEQ ID NO: 1 - DNA, 1065 bp, Artificial sequence
  Human VEGF-D
atgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttga

SEQ ID NO: 2 - protein, 354 aa, Artificial sequence
  Fill-length human VEGF-D
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 3 - protein, 354 aa, Artificial sequence; identical to SEQ ID NO: 2
  Wild-type full-lenght human VEGF-D

SEQ ID NO: 4 - protein, 225 aa, Artificial sequence
  VEGF-D SSTS.?C
MVLASSTTSIHTMLLLLLMLFHLGLQASISARQDYKDDDDKTRNEHGPVKRSSQSTLERSEQQIRAASSLEELLRITHSEDWKLWRCRLRLKSFTSMDSRSASHSSTSFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRR

SEQ ID NO: 5 - protein, 309 aa, Artificial sequence
  VEGF-D N.IISS
MVLASSTTSIHTMLLLLLMLFHLGLQASISARQDYKDDDDKTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIISSSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 6 - protein, 8 aa, Artificial sequence
  Synthetic octapeptide
DYKDDDDK

SEQ ID NO: 7 - protein, 4 aa, Artificial sequence
  Synthetic peptide
  misc_feature (2)..(2): Xaa can be any naturally occurring amino acid
  misc_feature (3)..(3): X=K or R
RXXR

SEQ ID NO: 8 - DNA, 12 bp, Artificial sequence
  Synthetic primer
cggtccactagg

SEQ ID NO: 9 - DNA, 12 bp, Artificial sequence
  Synthetic primer
tcctccacttcc

SEQ ID NO: 10 - DNA, 40 bp, Artificial sequence
  R85S mutation pEFsBOS-VEGF-D
gcatcccatcggtccacttcctttgcggcaactttctatg

SEQ ID NO: 11 - DNA, 40 bp, Artificial sequence
  R85S mutation pEFsBOS-VEGF-D
catagaaagttgccgcaaaggaagtggaccgatgggatgc

SEQ ID NO: 12 - DNA, 36 bp, Artificial sequence
  R88S mutation pEFsBOS-VEGF-Drsts
ctcgctcagcatcccattcctccacttcctttgcgg

SEQ ID NO: 13 - DNA, 36 bp, Artificial sequence
  R88s mutation pEFsBOS-VEGF-Drsts
ccgcaaaggaagtggaggaatgggatgctgagcgag

SEQ ID NO: 14 - DNA, 12 bp, Artificial sequence
  Synthetic primer
attatcagaaga

SEQ ID NO: 15 - DNA, 12 bp, Artificial sequence
  Synthetic primer
attatcagcagc

SEQ ID NO: 16 - DNA, 12 bp, Artificial sequence
  Synthetic primer
attatctccagc

SEQ ID NO: 17 - DNA, 40 bp, Artificial sequence
  Synthetic primer
ccatactcaattatcagcagctccatccagatccctgaag

SEQ ID NO: 18 - DNA, 40 bp, Artificial sequence
  Synthetic primer
cttcagggatctggctggagctgctgataattgagtatgg

SEQ ID NO: 19 - DNA, 40 bp, Artificial sequence
  Synthetic primer
ccatactcaattatctccagctccatccagatccctgaag

SEQ ID NO: 20 - DNA, 40 bp, Artificial sequence
  Synthetic primer
cttcagggatctggatggagctggagataattgagtatgg

SEQ ID NO: 21 - DNA, 44 bp, Artificial sequence
  Synthetic primer
gcgtctagactagtgctagctcatcatcttctgataattgagta

SEQ ID NO: 22 - DNA, 20 bp, Artificial sequence
  Synthetic primer
ccagcttggcacttgatgta

SEQ ID NO: 23 - DNA, 27 bp, Artificial sequence
  Synthetic primer
gcgacgcgttttgcggcaactttctat

SEQ ID NO: 24 - DNA, 21 bp, Artificial sequence
  Synthetic primer
gatggggaacactgctgttta